(19) United States
(12) Patent Application Publication
(10) Pub. No.: US 2023/0372645 A1
(43) Pub. Date: Nov. 23, 2023

(54) DEVICE FOR DISPENSING A FLUID PRODUCT

(71) Applicant: APTAR FRANCE SAS, LE NEUBOURG (FR)

(72) Inventors: Fabien ADAM, AVIRON (FR); Mickaël BAILLIE, ACQUIGNY (FR)

(73) Assignee: APTAR FRANCE SAS, LE NEUBOURG (FR)

(21) Appl. No.: 18/029,940

(22) PCT Filed: Oct. 4, 2021
(86) PCT No.: PCT/FR2021/051707

§ 371 Date: Apr. 3, 2023

(30) Foreign Application Priority Data
Oct. 6, 2020 (FR) FR2010192

(51) Int. Cl. A61M 15/00; G01F 11/36; G01F 15/00

(52) U.S. Cl. CPC A61M 15/0066; A61M 15/0021; A61M 15/002; G01F 11/36; G01F 15/005; A61M 2205/50

(57) ABSTRACT

The invention concerns a device for dispensing a fluid product comprising a body (10), a mouthpiece (20), a reservoir (100) containing a fluid product and a propellant gas slidably mounted in said body, a valve (200) comprising a valve member (210) assembled on said reservoir, the outlet from said valve member being connected to said mouthpiece by a channel (15), and an electronic unit (400), wherein:
said valve is continuous, with an actuation position in which the reservoir is connected to the outlet from said valve member,
said body comprises a cover (30) which, in the closed position, urges said valve member into its actuation position,
said device comprises a metering system (300) controlled by said electronic unit, said metering system comprising a shutter (310) which is closed at rest and which, during actuation, is displaced into its open position for a predetermined time in order to dispense a dose of fluid product.

The present invention relates to a device for dispensing a fluid product, and more particularly to an oral inhaler device of the aerosol type.

Oral inhaler devices are well known in the art. The principal advantage of this type of device is that the product is dispensed with a propellant gas so as to guarantee that the product is properly dispensed into the respiratory tract. In general, these devices comprise a metering valve adapted to dispense the same dose of fluid product each time it is actuated. In addition, each dose is dispensed in the same manner, without the possibility of modulating the dispensing parameters.

Documents U.S. Ser. No. 10/589,052 B1, US 2019/167939 A1 and US 1 055 6072 B2 describe the use of an electronically controlled microvalve to improve and guarantee the metering accuracy of a metering valve each time it is actuated. Document US 2005/081846 describes a prior art device.

The object of this invention is to provide a fluid product dispensing device that does not have the above mentioned disadvantages.

Another object of the present invention is to provide a device for dispensing a fluid product which improves operational reliability by guaranteeing effective actuation upon each inhalation.

Another object of the present invention is to provide a device for dispensing a fluid product which can be used to modify the metering and/or dispensing characteristics and/or parameters each time it is actuated.

Another object of the present invention is to provide a device for dispensing a fluid that is simple and inexpensive to manufacture and to assemble.

The present invention therefore provides a device for dispensing a fluid product comprising a body provided with a mouthpiece, a fluid product reservoir containing a fluid product and a propellant gas being mounted to slide axially in said body, a valve comprising a valve member being assembled on said reservoir in order to dispense said fluid product, said valve member being displaceable between a rest position and an actuating position, the outlet from said valve member being connected to said mouthpiece by a channel, said device further comprising an electronic unit, in which:

said valve is a continuous valve in which, in the actuation position of the valve member, the reservoir is permanently connected to the outlet from said valve member,

said body comprises a cover which can be displaced between an open position and a closed position in which it urges said valve member into its actuation position;

said device comprises a metering and dispensing system controlled by said electronic unit, said metering and dispensing system comprising a shutter member which can be displaced between a closed position in which it closes said channel and an open position in which it opens said channel, said shutter member being at rest in the closed position and being displaced into its open position for a predetermined time during actuation of the device in order to dispense a dose of fluid product.

Advantageously, said cover is mounted to pivot on said body about a pivot axis between its open and closed positions, an axial projection being provided in the base of said cover, said axial projection co-operating in the closed position of said cover with a base of said reservoir in order to axially displace said valve member into the actuation position.

Advantageously, releasable securing means are provided in order to secure said cover on said body and to hold it in the closed position.

Advantageously, said electronic unit stores metering and/or dispensing characteristics and/or parameters on the basis of which said metering and dispensing system is controlled.

Advantageously, said metering and/or dispensing characteristics and/or parameters can be modified.

Advantageously, said metering and/or dispensing characteristics and/or parameters comprise:
the volume of the dispensed dose,
the dispensing duration,
the flow rate of the fluid during dispensing.

Advantageously, said shutter member is controlled by a motor or by a solenoid valve which is controlled by said electronic unit.

Advantageously, said shutter member is a piston.

Advantageously, said electronic unit comprises reservoir identification means, in a manner such that when a reservoir is put into place in said body, said electronic unit identifies said reservoir and its contents, and adapts the metering characteristics and parameters as a function of the type of reservoir.

Advantageously, said electronic unit comprises a screen.

Advantageously, said electronic unit comprises an accelerometer for verifying that the user has in fact shaken the device before use.

Advantageously, said electronic unit comprises a position sensor for verifying the position of the device at the time of use.

Advantageously, the electronic unit comprises a dose counter for indicating to the user the number of doses that have been dispensed or which remain to be dispensed in the reservoir.

These and other characteristics and advantages will appear more clearly from the following detailed description made with reference to the accompanying drawings given by way of non-limiting examples, and in which:

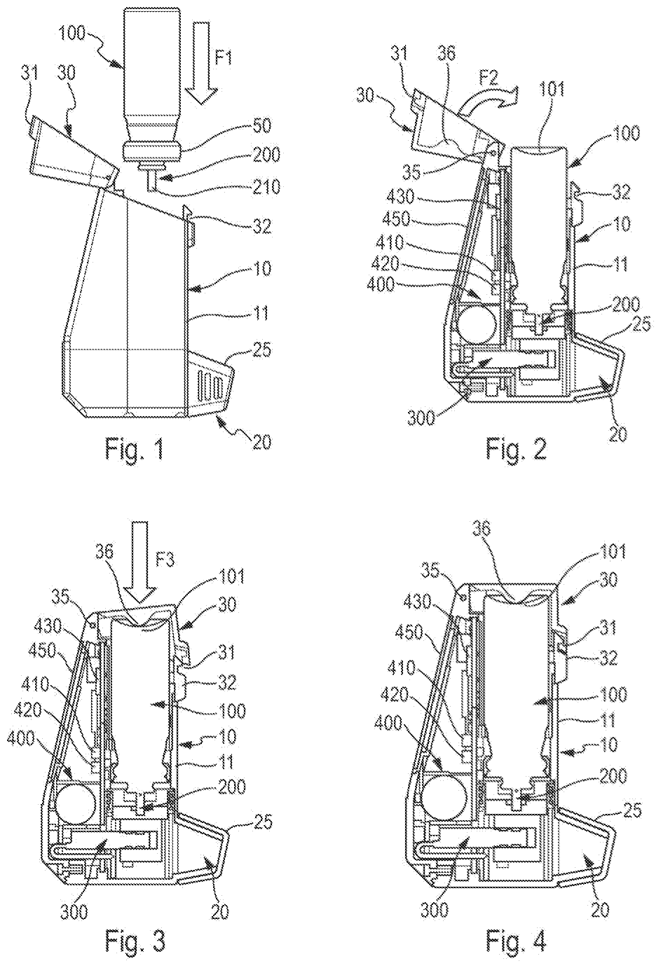

FIG. 1 is a diagrammatic side view of a fluid product dispensing device in accordance with an advantageous embodiment before assembling the reservoir with the body,

FIG. 2 is a diagrammatic cross sectional view of the device of FIG. 1 after assembling the reservoir with the body and before closing the cover,

FIG. 3 is a diagrammatic view similar to that of FIG. 2, while the cover is being closed,

FIG. 4 is a diagrammatic view similar to that of FIG. 3, after the cover has been closed,

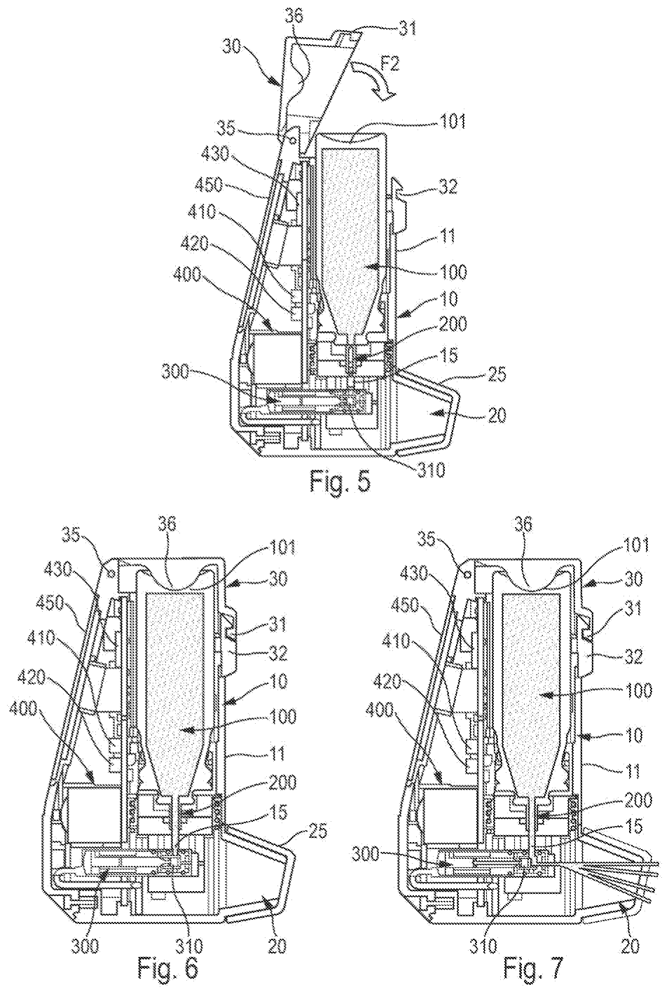

FIG. 5 is another diagrammatic cross-sectional view of the device of FIG. 1, after assembling the reservoir with the body and before the cover has been closed,

FIG. 6 is a view similar to that of FIG. 5, after the cover has been closed and before actuating the dispensing system, and

FIG. 7 is a view similar to that of FIG. 6, during actuation of the dispensing system.

In the description, the term “axial” refers to the central vertical axis A of the valve shown in FIG. 1. The terms “proximal” and “distal” are relative to the mouthpiece.

The figures show an advantageous embodiment of the invention, but naturally one or more of the component parts described below could be made in some other way, while providing functions that are similar or identical.

In known manner, a device for dispensing a fluid product comprises a body 10 provided with a mouthpiece 20, a product reservoir 100 containing a fluid product and a valve 200. The present invention also provides a system 300 for metering and dispensing a dose of fluid product each time it is actuated which is controlled by an electronic unit 400.

The body 10 may be made as a single piece or out of a plurality of parts that are assembled together. It comprises a cylindrical body part 11 receiving the reservoir 100.

A cover 30 is provided to cooperate, in the closed position, with the base 101 of the reservoir 100. Advantageously, the cover 30 is mounted to pivot on the body 10 about a pivot axis 35 between an open position and a closed position. In the open position, visible in FIGS. 1, 2, and 5, the reservoir 100 may be inserted into or removed from the body 10. In the closed position, visible in FIGS. 4, 6 and 7, an axial projection 36 provided in the base of the cover 30 cooperates with the base 101 of the reservoir 100 in order to displace said reservoir 100 axially in the body 10.

Releasable securing means are preferably provided for securing the cover 30 on the body 10 and for holding it in the closed position. These securing means may comprise a hook 32 fastened to the body 10 and co-operating with a snap fastening endpiece 31 of the cover which is complementary to the hook 32, in order to provide releasable fastening, for example by snap fastening. Other securing means may also be envisaged.

The mouthpiece 20 defines a dispensing orifice through which the user inhales while the device is being used. The mouthpiece 20 may be made integrally with the body 10. A removable protective cap 25 may be provided on said mouthpiece 20, in particular while it is being stored, that the user removes before use.

The body 10 contains a reservoir 100 which contains the product to be dispensed and a propellant gas, such as an HFA type gas, a valve 200 being mounted on said reservoir 100 for dispensing the product. The valve 200 comprises a valve body (not shown) and a valve member 210 which, during actuation, is axially displaceable with respect to said valve body, and thus with respect to said reservoir 100, between a rest position and an actuation position.

In accordance with the invention, this valve 200 is a continuous valve, i.e. a valve in which the contents of the reservoir 100 are permanently connected to the outlet from the valve member 210 when the valve member is in the actuation position, by being pushed axially into the valve body.

This valve 200 may be of any appropriate type. It is secured to the reservoir 100 via a securing element 50, preferably a crimped cap, preferably with a neck gasket interposed therebetween (not shown).

The present invention therefore does not require a metering valve, which is much more complex and therefore expensive to manufacture and to assemble than a continuous valve.

Advantageously, during actuation, the valve member 210 is stationary relative to the body 10, and it is the reservoir 100 that is moved axially relative to the body 10 between a distal position, which is the rest position, and a proximal position, which is the actuation position.

When the reservoir 100 is inserted into the body 10 and the cover 30 is in the closed position, the valve member 210 is displaced into its actuation position in a manner such that the valve 200 is permanently open.

The outlet orifice of the valve member 210 of said valve 200 is connected via a channel 15 to said mouthpiece 20 through which the user inhales the fluid which is dispensed.

The metering and dispensing system 300 is adapted to selectively close or open said channel 15. At rest, the channel 15 is closed, and when actuated by the user, the channel 15 is open for a predetermined duration, generating dispensing of a dose of fluid product.

This metering and dispensing system 300 is controlled by an electronic unit 400 which contains the predeterminable metering and/or dispensing characteristics and parameters. These characteristics and parameters may be predetermined and/or modified, either by the user or only by authorized personnel. These modifications and adjustments may be made either directly on the device, for example via a screen 450 of the electronic unit 400, or remotely via a wireless link.

The metering and dispensing system 300 comprises a shutter member 310 which can be displaced between an open position and a closed position of the channel 15. The displacement of the shutter member 310 is controlled by a motor or a solenoid valve controlled by the electronic unit 400.

In the example shown, the shutter member 310 is a piston, but it could be produced differently. The remainder of the description shall make reference to a piston.

An actuating button may be provided in order to enable the user to trigger dispensing of a dose. This button may be of any shape, for example formed on the body 10, or, in a variation, it may be integrated into a screen 450 of the electronic unit 400. Its actuation by the user triggers the electronic unit 400, which will then manage the metering and dispensing system 300 in accordance with the metering and/or dispensing characteristics and parameters stored in the electronic unit 400. Advantageously, the electronic unit 400 may comprise means for identifying the user, for example by fingerprints or by facial recognition, in particular in order to avoid unauthorized actuation of the device.

Thus, the metering is no longer defined by the valve 200, but solely by the metering and dispensing system 300. This makes it possible to program or predict variations in doses as a function of the needs or the users, and/or to modify the dose, if necessary.

Advantageously, the electronic unit 400 comprises means for identifying the reservoir 100, in a manner such that when the reservoir 100 is put into place in the body 10, the electronic unit 400 identifies the reservoir and its contents and adapts the metering characteristics and parameters as a function of the type of reservoir. Thus, for a reservoir of type A containing a fluid A, the opening time for the piston 310 may be 3 seconds, whereas for a reservoir of type B containing a fluid B, this opening time may be 0.5 seconds.

The characteristics and parameters of the dose and/or of the distribution may in particular comprise:
the volume of the dispensed dose,
the dispensing duration, typically between 0.15 and 3 seconds,
the flow rate of the fluid during dispensing.

The electronic unit 400 comprises all the electronic elements necessary to control the metering and dispensing system 300. In particular, it comprises an energy source such as a battery, a storage battery or a rechargeable battery, a programmable microprocessor, a memory. Advantageously, a screen 450 may be associated with the electronic unit 400 in order to display information and optionally to allow interaction with the user. Other components may also be provided, depending on requirements. As an example, an accelerometer 410 is advantageously provided in order to verify that the user has in fact shaken the device before use. In the absence of such a shaking action, the metering and dispensing system 300 remains locked. In addition, a position sensor 420 is advantageously provided in order to verify the position of the device at the time of use. If the device is not in a correct position of use, the metering and dispensing system 300 remains locked. A dose counter 430 is also advantageously provided to indicate to the user, for example via the screen 450, the number of doses which have been dispensed or which remain to be dispensed in the reservoir 100. These various elements are not described in greater detail here since they are conventional electronic components which are well known to the person skilled in the art.

The electronic unit 400 and its various components are shown only very diagrammatically in the figures, and could be produced and arranged differently in the device.

An advantage of the present invention is that the body 10, the metering and dispensing system 300 and the electronic unit 400 can be reused. Thus, only the reservoir 100 and the valve 200 are discarded after use. After cleaning the channel 15 and the mouthpiece 20, a fresh reservoir 100 with its valve 200 can be assembled with the body 10.

Operation of the device shown in the figures is as follows:
after having inserted the reservoir 100 with its valve 200 into the body 10, in the direction of the arrow F1 in FIG. 1, the user closes the cover 30 in the direction of the arrow F2 in FIGS. 2 and 5;
the locking of the cover 30 on the body 10 in the direction of the arrow F3 in FIG. 3 causes the axial displacement of the reservoir 100 with respect to the valve member 210 of the valve 200, bringing said valve into an open position in which the reservoir is permanently connected to the outlet from the valve member 210;
since the piston 310 closes the channel 15, the fluid cannot escape from said channel 15 beyond said piston 310;
when the user is ready to use the device, they shake it correctly, remove the removable protective cap 25 from the mouthpiece 20 and place the mouthpiece in their mouth in the correct orientation;
they then actuate the device, for example by pressing on an actuating button, which will trigger the electronic unit 400; after validation of shaking and the correct orientation of the device, the electronic unit will control the metering and dispensing system 300 to displace the piston 310 from its closed position to its open position for a predetermined time; this will dispense a dose of fluid product through the mouthpiece for inhalation by the user;
after said predetermined time, the metering and dispensing system 300 will automatically return the piston 310 to its closed position; the user can then remove the device from their mouth and replace the removable protective cap 25 on the mouthpiece, optionally cleaning it; the device is then ready for the next use.

The present invention is of application to all types of formulations and to all types of active products contained in the reservoir. Similarly, the propellant gas may be of any type, in particular of the HFA type, for example HFA-134a and/or HFA-227 and/or HFA-152a, and/or HFO1234ze.

The present invention has been described with reference to an advantageous embodiment, but naturally any modification could be applied thereto by the person skilled in the art, without going beyond the scope of the present invention, as defined by the accompanying claims.

1. A device for dispensing a fluid product, comprising a body (provided with a mouthpiece, a product reservoir containing fluid product and a propellant gas being mounted to slide axially in said body, a valve comprising a valve member being assembled on said reservoir in order to dispense said fluid product, said valve member being displaceable between a rest position and an actuation position, the outlet from said valve member being connected to said mouthpiece by a channel, said device further comprising an electronic unit, characterized in that:
said valve is a continuous valve in which, in the actuation position of the valve member, the reservoir is permanently connected to the outlet from said valve member,
said body comprises a cover which can be displaced between an open position and a closed position in which it urges said valve member into its actuation position;
said device comprises a metering and dispensing system controlled by said electronic unit, said metering and dispensing system comprising a shutter member which can be displaced between a closed position in which it closes said channel and an open position in which it opens said channel, said shutter member being at rest in the closed position and, when the device is actuated, being displaced into its open position for a predetermined time in order to dispense a dose of fluid product.

2. The device as claimed in claim 1, wherein said cover is mounted to pivot on said body about a pivot axis between its open and closed positions, an axial projection being provided in the base of said cover, said axial projection cooperating in the closed position of said cover with a base of said reservoir in order to axially displace said valve member into the actuation position.

3. The device as claimed in claim 2, wherein releasable securing means are provided in order to secure said cover on said body and to hold it in the closed position.

4. The device as claimed in claim 1, wherein said electronic unit stores metering and/or dispensing characteristics and/or parameters on the basis of which said metering and dispensing system is controlled.

5. The device as claimed in claim 4, wherein said metering and/or dispensing characteristics and/or parameters can be modified.

6. The device as claimed in claim 1, wherein said metering and/or dispensing characteristics and/or parameters comprise:
the volume of the dispensed dose,
the dispensing duration,
the flow rate of the fluid during dispensing.

7. The device as claimed in claim 1, wherein said shutter member is controlled by a motor or a solenoid valve controlled by said electronic unit.

8. The device as claimed in claim 1, wherein said shutter member is a piston.

9. The device as claimed in claim 1, wherein said electronic unit comprises reservoir identification means, in a manner such that when placing a reservoir in said body, said electronic unit identifies said reservoir and its contents and adapts the metering characteristics and parameters as a function of the type of reservoir.

10. The device as claimed in claim 1, wherein said electronic unit comprises a screen.

11. The device as claimed in claim 1, wherein said electronic unit comprises an accelerometer for verifying that the user has in fact shaken the device before use.

12. The device as claimed in claim 1, wherein said electronic unit comprises a position sensor for verifying the position of the device at the time of use.

13. The device as claimed in claim 1, wherein said electronic unit comprises a dose counter for indicating to the user the number of doses which have been dispensed or which remain to be dispensed in the reservoir.
